(19) United States
(12) Patent Application Publication
(10) Pub. No.: US 2013/0261501 A1
(43) Pub. Date: Oct. 3, 2013

(54) Digital Stethoscope

(71) Applicant: Robert Winston Carter, (US)

(72) Inventor: Robert Winston Carter, (US)

(21) Appl. No.: 13/435,669

(22) Filed: Mar. 30, 2012

(51) Int. Cl. A61B 7/04

(52) U.S. Cl. 600/586

(57) ABSTRACT

A digital stethoscope has an earpiece microphone (4) connected acoustically with flexible tubing to a Precordial Hockey-Puck sensor (1) and connected electronically to a Pro-audio Computer (8) with real-time software to display, control monitoring and record vital signs of a patient undergoing medical procedures. The patient is acoustically isolated from the computer. The user interface can select or de-select optional functional real-time code blocks.

### BACKGROUND

1. Field of the Invention

This invention relates to the medical monitoring, display and recording of vital acoustic sounds from a human patient or animal subject as when undergoing anesthesia and other procedures while in an operating room or equivalent setting, such as a field hospital or research lab. The sounds of interest include those from the patient heart, lungs and other structures.

2. Description of Prior Art

A physician responsible for the conduct of anesthesia often uses a primitive Precordial Stethoscope consisting of a Hockey-Puck acoustic sensor (1) with flexible tubing ending in a simple ear piece. While the primitive Precordial Stethoscope works, it is limited to the auditory capability of the doctor while also acting like a leash. The Digital Stethoscope was designed to enhance the Stethoscope with digital information technology including real-time amplification, graphical waveform monitoring, controlled acoustic monitoring and audio data file recording. Durand et. al, U.S. Pat. No. 005,602,924A did work toward using electronics with a stethoscope. Saltzman, U.S. Pat. No. 4,777,961 also did work toward an electronic stethoscope.

### SUMMARY OF THE INVENTION

### Object and Advantages

Object of patentable novelty and utility taught by this invention are to provide a Digital Stethoscope that:

Has a Hockey-Puck acoustic sensor (1) connected with flexible tubing to a microphone (3).

Has the microphone (3) connected electrically through protected tubing to a Pro-Audio computer (8).

Has real-time computer software to capture the microphone (3) output.

Has real-time computer software (9) to duplicate the microphone (3) signal to permit recording into an audio file (11).

Has real-time computer software (9) to duplicate the microphone (3) signal to permit graphical waveform display (12).

Has real-time computer software (9) to duplicate the microphone (3) signal to permit headphone or speaker monitoring (14) with user-selected amplitude limiting (13).

The invention accomplishes the objectives: with the Hockey-Puck acoustic sensor (1) temporarily bonded to the patient as in normal practice, the physician can listen to the vital signs with headphones, look at the acoustic waveforms on a normal or special computer display while the computer is recording the microphone output into a standard audio file for later review or teaching and related medical functions. Since the system is based heavily on real-time software control, it is possible to upgrade or customize the software blocks as needed to fine tune for special application areas.

### BRIEF DESCRIPTION OF FIGURES

The invention is described by appended claims in relation to a preferred embodiment with reference to the following figures that are explained briefly as follows:

FIG. 1 is a view of the microphone assembly in the Digital Stethoscope that captures acoustic information from the patient. The microphone assembly provides acoustic isolation of the patient from the electronics used downstream in a computer.

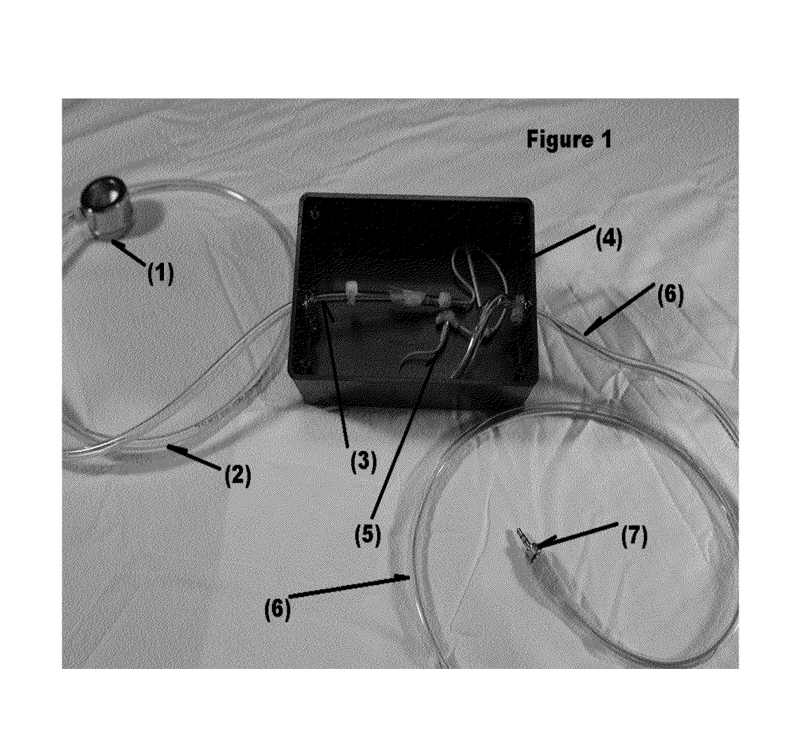

FIG. 2. is a block diagram or graph of the major software elements in the Digital Stethoscope.

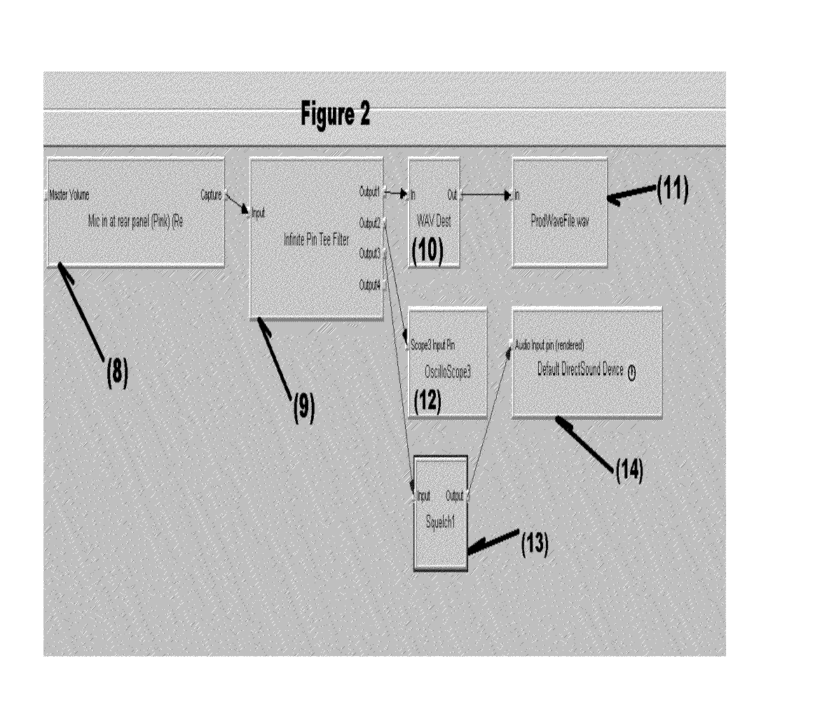

FIG. 3 is a view of the user interface to the Digital Stethoscope allowing the physician to pick a limit or squelch level, start the monitoring and recording and later stop the session.

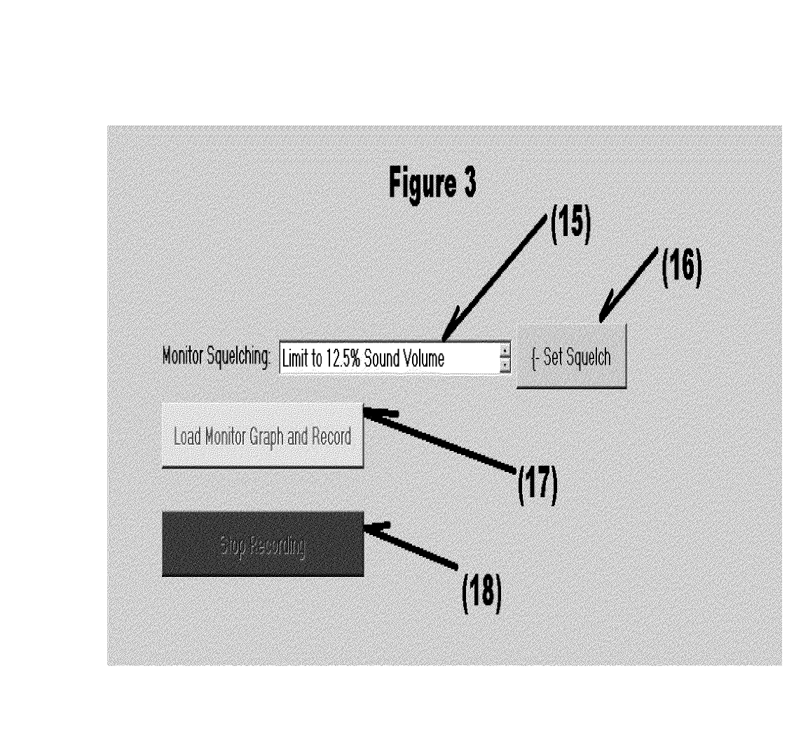

### DESCRIPTION OF PREFERRED EMBODIMENT

### Reference Numerals

Listed numerically below with reference to the figures are terms used to describe features of the invention. The terms and numbers assigned to them are used throughout this description.

1. Acoustic Hockey-Puck Precordial Sensor.

2. Acoustic Tubing Sensor (1) Input.

3. Earpiece-Type Microphone inside Acoustic Tubing.

4. Earpiece Microphone Base (3).

5. Earpiece Microphone (3) Electrical Output.

6. Earpiece Microphone Electrical (3) Cable inside Tubing.

7. Earpiece Microphone (3) Cable Jack for Computer Input.

8. Software Microphone Input Block.

9. Software Tee Filter Signal Duplication Block.

10. Software PCM Wave File Destination Block.

11. Software Wave File Output Block.

12. Software Acoustic Waveform Graphical Output Block.

13. Software Squelching or Amplitude-Limiting Output Block.

14. Software Squelched Headphone or Speaker Output Block.

15. Squelch Level Picklist for User Interface.

16. Button to Pick Squelch Level for User Interface and allow session start.

17. Button to Start Monitoring and Recording for User Interface.

18. Button to Stop Recording and Monitoring.

The preferred embodiment of the system is to mount the computer and display device(s) on a portable cart supported with casters so staff can move the unit as needed in patient care. Various small computers are now available with Pro-audio capability, which normally means operation without fans and with 24-bit data recording sampling of at least 44 kHz or more. The development system does all the functions listed above using about 20% of the real-time resources of the computer.

What is claimed:

1. A Digital Stethoscope comprising:
One or more Hockey-Puck acoustic sensors each connected with a flexible first tubing to the input side of a microphone contained in the first tubing with the output electrical side cable of the microphone contained in a second tubing to connect to a digital computer capable of audio input.

2. A Digital Stethoscope of claim 1 wherein:
The digital computer is equipped with real-time software that can capture microphone outputs, duplicate the microphone audio output into streams producing graphical waveform displays, with audio file capture and amplitude-controlled monitoring for the end user. The software can contain optional code blocks for network transmission and other functions for unique patient-care requirements.

3. A Digital Stethoscope of claim 2 wherein:
The digital computer is equipped with real-time software to permit the end user to select from choices of amplitude-controlled monitoring, start a monitoring and recording session then later stop the session. The user interface can permit selection or de-selection of optional software code blocks within the real-time software to adapt to unique patient-care requirements.
